(19) United States
(12) Patent Application Publication
(10) Pub. No.: US 2021/0179856 A1
(43) Pub. Date: Jun. 17, 2021

(54) CARBON-COATED BiOCl PIGMENTS

(71) Applicant: MERCK PATENT GMBH, Darmstadt (DE)

(72) Inventors: Adeliene SCHMITT, Ludwigshafen (DE); Padma KAVIRATNA, Philadelphia, PA (US); Udo GUMSHEIMER, Darmstadt (DE); Mark TELLEFSEN, Gibbstown, NJ (US); Qingmin CHENG, Philadelphia, PA (US)

(73) Assignee: MERCK PATENT GMBH, DARMSTADT (DE)

(21) Appl. No.: 17/117,968

(22) Filed: Dec. 10, 2020

(30) Foreign Application Priority Data
Dec. 11, 2019 (DE) 102019008593.0

(51) Int. Cl. C09C 3/06; A61K 8/20

(52) U.S. Cl. CPC C09C 3/063; A61K 8/20; A61K 2800/651; A61K 2800/621; C09C 2200/301; C09C 2200/1087; C09C 2200/401; C09C 2200/505; C09C 2200/307; A61K 2800/43

(57) ABSTRACT

BiOCl pigments coated with an amorphous carbon. A process for the preparation of the pigments. And use of the pigments prepared in this way, inter alia in paints, coatings, printing inks, plastics and cosmetics.

The present invention relates to flake-form BiOCl pigments coated with amorphous carbon, to a process for the preparation of the pigments, and to the use of the pigments prepared in this way, inter alia in paints, coatings, printing inks, plastics and in cosmetics.

Metal-effect pigments have been employed for many years in coatings for the generation of metallic effects, for example in printing inks or in metallic finishes for automobiles. Classical metal-effect pigments consist of flake-form metal particles, whose optical action is based on the directed reflection of incident light at the surface, which is ideally formed flat and planar, of the metal particles, which are aligned parallel to the surface in the respective application medium.

The most frequently employed types of metal-effect pigment worldwide consist of aluminium, in addition also copper and copper/zinc pigments or also zinc pigments. Shape, thickness, size and surface quality of the metal pigments are determined by the preparation process.

Thin metal-effect pigments, in particular aluminium flakes, are employed if it is desired to achieve a so-called “liquid-metal” effect in coatings, such as, for example, automotive paints. Aluminium pigment flakes are very expensive as a consequence of their production and, owing to their intrinsically reactive surface, have to be handled with considerable safety measures in all applications. The aluminium pigment flakes are dispersed in coating systems and subsequently applied to the surface to be coated by means of spray application or electrostatic application. In order to achieve the liquid-metal effect, a 7-coat application is generally necessary. After application of each paint coat comprising aluminium pigment flakes, the applied layer first has to be dried in order that orientation of the pigments plane-parallel to the coated surface is achieved. Owing to these high raw materials and handling costs for the pigment and the complex special coating process (7-layer structure including separate drying steps), the liquid-metal effect finish is, for the automotive sector, restricted to expensive premium products/premium automobiles.

For special finishes in the automotive sector, interference/colour-flop pigments, which are less-expensive and are not hazardous from a safety point of view, are therefore often employed. However, effect pigments, such as, for example, interference pigments and colour-flop pigments, based on inorganic substrates, are several times thicker than the intrinsically thin metal pigment flakes and are therefore generally unsuitable for a liquid metal-effect finish. This is made more difficult by the fact that the pigment particle surface of interference pigments has, as a consequence of the production process, surface roughness, which functions as centres of scattering for diffuse light. The incident light therefore cannot be reflected at the surface, as compared with an ideally smooth (mirror) surface. A finish comprising interference pigments consequently does not exhibit a pronounced liquid-metal effect.

A finish exhibits the liquid-metal effect if the outer appearance is similar to a silver-coloured metal in a liquid melt or a metal coating from a metallisation bath. The finish is perceived by the observer as texture-free, absolutely homogeneous and having a metallic silver lustre. Furthermore, the surface is perceived as extremely bright from every viewing direction, without lustre or brightness differences, such as, for example, a matt/gloss change or a bright/dark flop as a consequence of a changing viewing angle. It is only due to the plane-parallel orientation of the pigments in each paint coat that the final liquid-metal finish appears homogeneous and texture-free and is reminiscent of a “liquid” metal surface.

The requirement for freedom from texture means that the pigments dispersed in the coating system are in flake form and have a thickness that is not greater than the thickness of the coating film. The pigments should ideally have a thickness of <60 nm.

An effect pigment having very high brightness is flake-form bismuth oxychloride (BiOCl). The metallic silver-white lustre and the physiological harmlessness have made BiOCl indispensable in decorative cosmetics. The low light stability—short-wave light colours BiOCl grey—the rapid settling behaviour and low mechanical resilience are disadvantages of BiOCl pigments in industrial areas of application, such as, for example, industrial coatings, automotive paints, inks, plastics, etc. In order to increase the hiding power, BiOCl pigments are frequently admixed with a black additive, such as, for example, carbon black or black iron oxide. Although the mixing of the additives increases the hiding power, it leads, however, to further disadvantages, such as, for example, a yellowish appearance, separation phenomena, stability problems of a physical mixture, magnetism on use of iron oxides.

There is therefore a need to utilise the high metallic lustre of flake-form BiOCl pigments for all applications and to eliminate the above-mentioned disadvantages. The object of the present invention is to provide light-stable and highly reflective flake-form BiOCl effect pigments having a metallic appearance which, besides the high lustre, also have a high hiding power, and can be processed easily in all application media, such as, for example, paints, printing inks, plastics and in cosmetics. The BiOCl pigment should furthermore enable the generation of a pronounced liquid-metal effect in a simple manner in paint finishes.

Surprisingly, it has been found that BiOCl pigments which have been coated with a thin amorphous layer of carbon exhibit a metallic appearance with high lustre and at the same time the UV stability of the pigments is significantly increased. Furthermore, the pigments having a metallic lustre exhibit a very high hiding power.

The invention relates to coated BiOCl pigments, characterised in that flake-form BiOCl pigments have been coated on the surface with an amorphous carbon layer.

The stable pigments according to the invention are not electrically conductive, have an attractive silvery interference colour and very high lustre and have a significantly higher hiding power compared with the uncoated BiOCl pigments.

The invention also relates to a process for the preparation of the BiOCl pigments according to the invention, and to the use thereof, inter alia in paints, coatings, plastics and in cosmetics. The invention also relates to a simple paint application process using the pigments according to the invention for the production of finishes which exhibit an attractive liquid-metal effect.

Suitable substrates for the pigment according to the invention are all flake-form BiOCl pigments known to the person skilled in the art in the form of powders. BiOCl pigments are frequently only commercially available in the form of pastes, for example as paste form in castor oil. The pastes comprising BiOCl pigments are unsuitable as substrate for the carbon coating.

The BiOCl flakes preferably have particle sizes of 1-30 μm, in particular 1-25 μm and very particularly preferably 5-15 μm.

The thickness of the BiOCl flakes is preferably 50-100 nm, in particular 60-80 nm and very particularly preferably 40-60 nm.

The particle size refers to the greatest dimension of the pigment flakes in length and width.

The volume-weighted D₅₀ value is preferably <25 μm, in particular 5-20 μm and very particularly preferably 5-10 μm.

The volume-weighted D₉₀ and D₅₀ values in each case indicate that 90 and 50 percent by volume respectively of the pigments in a loose pigment pile have a particle size that is below the said value.

The choice of the particle size of the BiOCl pigments according to the invention has an influence on the final properties, since a small particle size, together with a very high fines content, is crucial for an increased hiding power of the pigments in the respective application media, while the individual pigment particles as such are semi-transparent, i.e., only a moderate hiding power can be expected in the application.

The particle size and particle size distribution can be determined by various methods which are usual in the art. However, preference is given in accordance with the invention to the use of the laser diffraction method in a standard method by means of a Malvern Mastersizer MS 3000 from Malvern Instruments Ltd., UK. These methods have the advantage that particle size and particle size distribution can be determined simultaneously under standard conditions.

The particle size and thickness of individual particles can in addition be determined with the aid of SEM (scanning electron microscope) images, in which particle size and geometrical particle thickness can be determined by direct measurement. For the determination of average values, at least 1000 particles are evaluated individually and the results averaged.

The BiOCl pigments according to the invention preferably have an aspect ratio (ratio of length or width to thickness) of 10-600, preferably 60-400.

Suitable BiOCl flakes in the form of powders are commercially available, for example, from Merck KGaA under the trade names RonaFlair® LF-2000, RonaFlair® B-50, RonaFlair® ESQ, RonaFlair® Fines, RonaFlair® MTU, and from BASF SE under the trade name Mearlite® MBU.

A thin amorphous carbon layer is applied to the flake-form BiOCl pigments (=substrates). This amorphous carbon layer is not electrically conductive and completely covers the substrate. The carbon layer is a compact, closed layer on the substrate. The geometrical thickness of the carbon layer is preferably 1-5 nm, in particular 1-3 nm and very particularly preferably 1-2 nm. This carbon layer with a thickness of only a few nanometres significantly increases the hiding power of the transparent BiOCl pigments, but without adversely affecting the lustre and reflectivity of the substrates.

The proportion of amorphous carbon, based on the coated BiOCl pigments, is preferably 1-5% by weight, in particular 1-3% by weight and very particularly preferably 1-2% by weight, based on the mass of the coated pigment.

In the case of the application of conformal, compact, amorphous and homogeneous carbon layers to the particulate BiOCl substrates, each individual BiOCl flake must be covered with a thin carbon layer in order to avoid unwanted centres of scattering. The carbon coating of particulate systems is usually applied by a complex wet-chemical hydrothermal synthesis in the presence of high pressures and temperatures or achieved by chemical vapour deposition (CVD process) taking place in the thermal gas phase. The thin BiOCl flakes are, however, only thermally stable up to a temperature of at most 350-400° C. before decomposition of the BiOCl and the formation of Bi₂O₃ commences.

The coating of the flake-form BiOCl pigments with the carbon layer is preferably carried out in a reactor. Suitable reactors in which the process according to the invention can be carried out are both rotary tubular furnaces and also fluidised-bed reactors, preferably the latter. The coating process is carried out with the aid of a stream of carrier gas. During the coating, the BiOCl pigments are kept in motion.

The carrier gas employed is an inert gas. Examples of inert gases which may be mentioned are nitrogen and argon, with nitrogen preferably being employed.

The carbon for the carbon coating can originate from pulverulent or volatile, i.e. gaseous carbon sources (=C sources).

In the case of pulverulent C sources, the procedure employed is as follows: The BiOCl pigment is mixed externally with one or more pulverulent carbon-containing compounds with the aid of a suitable mixer or in the reactor. These compounds are preferably selected from mono-, di- and trisaccharides, preferably fructose, glucose (=dextrose), galactose, xylose, mannose, lactose, sucrose, maltose or mixtures thereof. In a preferred embodiment, sucrose in the form of powdered sugar is employed.

In the case of gaseous C sources, such as, for example, acetone or ethine (acetylene), the C source is preferably admixed with the carrier gas.

However, the carrier gas may also itself consist of the gaseous carbon-containing compound, which in this case takes on both the function of the carrier gas and also that of the gaseous carbon-containing compound.

The use of pulverulent C sources is preferred. In a very particularly preferred embodiment, the C source is powdered sugar.

The application of the individual thin amorphous carbon layers to the BiOCl flakes is therefore preferably carried out in a fluidised bed-based CVD process using reactive/thermally unstable carbon precursors and at mild reaction temperatures, preferably at 150-400° C., in particular at 200-350° C. and very particularly preferably at 180-250° C. Fluidised-bed reactors have proven successful owing to their excellent heat- and material-transport properties and their good scalability. The comparatively low reaction temperatures during the thermal decomposition of the carbon precursor or carbon precursors lead to deposition of amorphous carbon layers on the BiOCl flakes.

In a preferred embodiment, the dry BiOCl flakes are mixed physically with fine monosaccharides in particulate form, such as, for example, commercially available glucose (dextrose), fructose or galactose, and also with disaccharides, for example maltose or sucrose (powdered sugar), etc., in an external mixer, such as, for example, a Turbula or drum hoop mixer, in the course of so-called “dry particle” coating. The fine sugar particles, such as, for example, saccharide particles (=guest particles), remain adhering to the BiOCl pigment surface (=host particles) via van der Waals forces.

The preparation of the mixture of saccharides and BiOCl pigment flakes in a defined ratio does not necessarily have to be carried out as described above in an external mixer, but can also be achieved in a fluidised bed.

Application of an aqueous carbon precursor, such as, for example, an aqueous saccharide solution, directly onto the surface of the BiOCl flake in a spray dryer or via an evaporator is furthermore possible.

The amount of carbon precursor employed, based on the substrate, is preferably 1-5% by weight, in particular 2-5% by weight, and very particularly preferably 1-3% by weight. An oversupply of carbon precursor may result in undesired, solid by-products, which may impair the final appearance of the coated BiOCl pigments.

In a preferred embodiment, BiOCl flakes conditioned with saccharide particles are preferably brought to reaction in the fluidised-bed process.

Firstly, they are fluidised in a fluidised-bed reactor under an inert-gas atmosphere. The mixing of the particles is achieved by the inert fluidisation gas, and can be improved by the use of fluidisation supports, such as vibrations, etc. In this way, agglomeration of the particles and channel formation within the fluidised bed is also reduced. Depending on the target thickness of the carbon layer, the saccharide used is decomposed thermally in a corresponding amount. The requisite heat is introduced into the fluidised BiOCl/disaccharide bed via the fluidised-bed reactor wall. After a certain reaction time of 5 to 30 min. and at reaction temperatures of 180-250° C., the CVD reaction or pyrolysis of the saccharide has advanced sufficiently that the BiOCl pigment flakes to be coated have been coated conformally and pinhole-free with a thin amorphous carbon layer.

With constant supply of inert gas, the coated BiOCl pigment flakes are cooled to room temperature and finally removed from the fluidised bed.

The carbon layer thickness is determined by the amount of carbon precursor, e.g., the amount of saccharide added.

The morphology of the deposited carbon is amorphous, which is confirmed with the aid of confocal Raman spectroscopy.

The present invention also relates to the use of the pigments according to the invention in paints, coatings, printing inks, coating compositions, plastics, films, in cosmetics, button pastes, in dry preparations and pigment preparations.

Due to their metallic appearance with high lustre and silvery interference colour and the extraordinarily high hiding power, the pigments according to the invention are very highly suitable for the pigmentation of application media. They are employed here in the same way as conventional interference pigments

The finely divided nature of the BiOCl pigments according to the invention facilitates very small layer thicknesses of the respective application medium, for example in the range 2-20 μm, without qualitative decreases in the hiding power of the coating having to be accepted. The pigments according to the invention are therefore suitable, in particular, for use in industrial coatings and automotive paints.

In paints, the pigments according to the invention are preferably employed in amounts of 20-50% by weight, based on the paint, and in effect finishes they are preferably employed in amounts of 8-30% by weight, based on the effect coating. The concentration employed here is dependent on the desired hue.

The BiOCl pigments according to the invention exhibit a pronounced liquid-metal effect in finishes.

It has been found, in particular, that the liquid-metal effect is particularly pronounced if, instead of the 7-step painting process known in the case of metal-effect pigments, a sequential three-step painting process, each with a subsequent drying step, is employed.

The carbon-coated BiOCl flakes are dispersed in either a water-based or solvent-based base coat. The suspension is subsequently applied sequentially to the surface to be painted. After each application, the base coat just applied is dried for 1-15 minutes, preferably 5 minutes, at a temperature of 60-90° C., preferably 80° C. The drying times depend on the paint system used. The second base coat is only applied after the drying step described. In total, this step is repeated three times. The finish is completed with a clear coat, into which, for example, one or more additives, such as, for example, UV stabiliser(s), can be incorporated.

LIDAR/RADAR sensors are already being employed today for driver assistance and are essential for autonomous driving. Aluminium-based pigments in automotive finishes have the disadvantage that aluminium-based pigments are impermeable for RADAR signals. The carbon-coated BiOCl pigments, for example, can be used as an alternative, since they are likewise distinguished by high reflectivity in the visible region and, as a consequence of the carbon coating, have improved an hiding power, and in addition are RADAR-permeable.

On use of the BiOCl pigments in paints and inks, all areas of application known to the person skilled in the art are possible, such as, for example, powder coatings, automotive paints, printing inks for gravure, offset, screen or flexographic printing and paints in outdoor applications. A multiplicity of binders, in particular water-soluble, UV-curing, but also solvent-containing types, for example based on acrylates, methacrylates, polyesters, polyurethanes, nitrocellulose, ethylcellulose, polyamide, polyvinyl butyrate, phenolic resins, melamine resins, maleic resins, starch or polyvinyl alcohol, are suitable for the preparation of printing inks. The paints can be water- or solvent-based and UV-curing paints, where the choice of the paint constituents is subject to the general knowledge of the person skilled in the art.

The BiOCl pigments according to the invention can likewise advantageously be employed for the production of plastics and films, for all applications known to the person skilled in the art. Suitable plastics here are all standard plastics, for example thermosets, elastomers and thermoplastics.

The BiOCl pigments according to the invention are also suitable for the preparation of flowable pigment preparations and dry preparations which comprise one or more pigments according to the invention, optionally further pigments or colourants, binders and optionally one or more additives. Dry preparations are also taken to mean preparations which comprise 0 to 8% by weight, preferably 2 to 8% by weight, in particular 3 to 6% by weight, of water and/or a solvent or solvent mixture. The dry preparations are preferably in the form of pearlets, pellets, granules, chips or briquettes and have particle sizes of about 0.2 to 80 mm.

The concentration of the BiOCl pigments according to the invention in the respective application medium is dependent on the colouration and lustre properties desired there and can in each case be selected by the person skilled in the art based on the usual recipes. The pigments according to the invention are preferably employed in plastics in amounts of 0.5-5% by weight (injection moulding: 0.5-1% by weight, thin films 8% by weight), based on the plastic,

in printing inks in concentrations of 10-25% by weight, based on the printing ink,
in cosmetics in concentrations of 0.01-100% by weight, preferably 2-30% by weight, based on the cosmetic formulation.

Although the BiOCl pigments according to the invention have attractive optical properties and can therefore be employed as the sole effect pigments in a very wide variety of applications, it is of course possible, and also advantageous depending on the application, to mix them where necessary with organic and/or inorganic colourants (in particular with white or coloured pigments) and to employ them together with the latter in an application, for example a coating.

The mixing ratios in the case of all mixtures described above are not limited so long as the advantageous properties of the pigments according to the invention are not adversely affected by the admixed foreign pigments. The pigments according to the invention can be mixed in any ratio with additives, fillers and/or binder systems that are usual in the application.

Suitable flake-form colourants are, preferably, pearlescent pigments, in particular based on natural or synthetic mica, SiO₂ flakes or Al₂O₃ flakes which are only coated with a metal-oxide layer, metal-effect pigments (for example on Al flakes, Fe flakes, bronzes), optically variable pigments (OVPs), liquid-crystal polymer pigments (LCPs) or holographic pigments.

Spherical colourants include, in particular, TiO₂, coloured SiO₂, CaSO₄, iron oxides, chromium oxides, carbon black, vegetable black, organic coloured pigments, such as, for example, anthraquinone pigments, quinacridone pigments, diketopyrrolopyrrole pigments, phthalocyanine pigments, azo pigments, isoindoline pigments. Needle-shaped pigments are preferably coloured glass fibres, α-FeOOH, organic coloured pigments, such as, for example, azo pigments, β-Phthalocyanine CI Blue 15.3, Cromophtal Yellow 8GN (Ciba-Geigy), Irgalith Blue PD56 (Ciba-Geigy), azomethine copper complex CI Yellow 129, Irgazine Yellow 5GT (Ciba-Geigy).

The BiOCl pigments according to the invention can furthermore be mixed with commercially available fillers. Fillers which may be mentioned are, for example, natural and synthetic mica, sodium potassium aluminium silicate, glass beads or glass powder, nylon powder, pure or filled melamine resins, talc, glasses, kaolin, oxides or hydroxides of aluminium, magnesium, calcium, zinc, BiOCl, barium sulfate, calcium sulfate, calcium carbonate, magnesium carbonate, carbon, and physical or chemical combinations of these substances.

There are no restrictions regarding the particle shape of the filler. In accordance with requirements, it can be, for example, flake-form, spherical, needle-shaped, crystalline or amorphous.

The use concentration and the mixing ratio of the BiOCl pigments according to the invention, in particular with organic and inorganic coloured pigments and dyes, of natural or synthetic origin, such as, for example, chromium oxide, ultramarine, spherical SiO₂ or TiO₂ pigments, are dependent on the application medium and the effect that is intended to be achieved.

The BiOCl pigments according to the invention can advantageously be employed in both decorative and care cosmetics, such as, for example, in lipsticks, lip gloss, eyeliner, eye shadows, rouge, sunscreen, pre-sun and after-sun preparations, make-ups, body lotions, bath gels, soaps, bath salts, toothpastes, hair gels, (volume) mascara, nail varnishes, compact powders, shampoos, loose powders and gels, etc.

The concentration of the BiOCl pigments according to invention in the application system to be pigmented is generally between 0.01 and 70% by weight, preferably between 0.1 and 50% by weight and in particular between 1.0 and 10% by weight, based on the total solids content of the system. It is generally dependent on the specific application and can be up to 100% in the case of loose powders. The use concentration of the BiOCl pigment according to the invention extends from 0.01% by weight in shampoo to 70% by weight in compact powder. In the case of a mixture of the BiOCl pigments according to the invention with spherical fillers, for example SiO₂, TiO₂, the concentration can be 0.01-70% by weight in the formulation. The cosmetic products, such as, for example, nail varnishes, lipsticks, compact powders, shampoos, loose powders and gels, are distinguished by their metallic lustre and their improved hiding power.

The BiOCl pigments according to the invention can also be combined in the formulations with cosmetic raw materials and assistants of any type. These include, inter alia, oils, fats, waxes, film formers, surfactants, antioxidants, such as, for example, vitamin C or vitamin E, stabilisers, odour enhancers, silicone oils, emulsifiers, solvents, such as, for example, ethanol or ethyl acetate or butyl acetate, preservatives and assistants that generally determine the technical application properties, such as, for example, thickeners and rheological additives, such as, for example, bentonites, hectorites, silicon dioxides, Ca silicates, gelatins, high-molecular-weight carbohydrates and/or surface-active assistants, etc.

Nanoscale dielectrics may likewise be admixed in order to improve the skin feel. Examples of admixtures of this type are Al₂O₃, SiO₂, ZnO or TiO₂, which are usually added to the formulation in amounts of 0.01-15%.

The formulations comprising the BiOCl pigments according to the invention can belong to the lipophilic, hydrophilic or hydrophobic type. In the case of heterogeneous formulations having discrete aqueous and non-aqueous phases, the pigment mixtures according to the invention may in each case be present in only one of the two phases or alternatively distributed over both phases.

The pH values of the formulations can be between 1 and 14, preferably between 2 and 11 and particularly preferably between 5 and 8.

No limits are set for the concentrations of the BiOCl pigments according to the invention in the formulation. They can be—depending on the application—between 0.001 (rinse-off products, for example shower gels) and 100% (for example lustre-effect articles for particular applications).

The BiOCl pigments according to the invention can furthermore also be combined with cosmetic active compounds. Suitable active compounds are, for example, insect repellents, inorganic UV filters, such as, for example, TiO₂, UV A/BC protection filters (for example OMC, B3, MBC), also in encapsulated form, anti-ageing active compounds, vitamins and derivatives thereof (for example vitamin A, C, E, etc.), self-tanning agents (for example DHA, erythrulose, inter alia), and further cosmetic active compounds, such as, for example, bisabolol, LPO, VTA, ectoin, emblica, allantoin, bioflavonoids and derivatives thereof.

Organic UV filters are generally incorporated into cosmetic formulations in an amount of from 0.5 to 10 percent by weight, preferably 1 to 8%, and inorganic filters in an amount of 0.1 to 30%.

The cosmetic preparations comprising the BiOCl pigment according to the invention may, in addition, comprise further conventional skin-protection or skin-care active compounds. These can in principle be all active compounds known to the person skilled in the art. Particularly preferred active compounds are pyrimidinecarboxylic acids and/or aryl oximes.

Cosmetic applications which may be mentioned are, in particular, the use of ectoin and ectoin derivatives for the care of aged, dry or irritated skin. Thus, European patent application EP-A-0 671 161 describes, in particular, that ectoin and hydroxyectoin are employed in cosmetic preparations, such as powders, soaps, surfactant-containing cleansing products, lipsticks, rouge, make-up, care creams and sunscreen preparations.

Application forms of the cosmetic formulations which may be mentioned are, for example: solutions, suspensions, emulsions, PIT emulsions, pastes, ointments, gels, creams, lotions, powders, soaps, surfactant-containing cleansing preparations, oils, aerosols and sprays. Further application forms are, for example, sticks, shampoos and shower preparations. Any desired customary vehicles, assistants and, if desired, further active compounds may be added to the preparation.

Ointments, pastes, creams and gels may comprise the usual vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the usual vehicles, for example lactose, talc, silica, aluminium hydroxide, calcium silicate and polyamide powder, or mixtures of these substances. Sprays may additionally comprise the usual propellants, for example chlorofluorocarbons, propane/butane or dimethyl ether.

Solutions and emulsions may comprise the usual vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerin fatty acid esters, polyethylene glycols and fatty acid esters of sorbitan, or mixtures of these substances.

Suspensions may comprise the usual vehicles, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the usual vehicles, such as alkali-metal salts of fatty acids, salts of fatty acid monoesters, fatty acid protein hydrolysates, isethionates, lanolin, fatty alcohols, vegetable oils, plant extracts, glycerin, sugars or mixtures of these substances

Surfactant-containing cleansing products may comprise the usual vehicles, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerin fatty acid esters, or mixtures of these substances.

Face and body oils may comprise the usual vehicles, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic compositions can be in various forms. Thus, they can be, for example, a solution, a water-free preparation, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatin, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions can be obtained in a conventional manner.

Further embodiments are oily lotions based on natural or synthetic oils and waxes, lanolin, fatty acid esters, in particular triglycerides of fatty acids, or oily/alcoholic lotions based on a lower alcohol, such as ethanol, or a glycol, such as propylene glycol, and/or a polyol, such as glycerin, and oils, waxes and fatty acid esters, such as triglycerides of fatty acids.

Solid sticks consist of natural or synthetic waxes and oils, fatty alcohols, fatty acids, fatty acid esters, lanolin and other fatty bodies.

If a preparation is formulated as an aerosol, the usual propellants, such as alkanes, fluoroalkanes and chlorofluoroalkanes, are generally used.

The cosmetic preparation may also be used to protect the hair against photochemical damage in order to prevent changes in colour, loss of colour or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a preparation in the form of a lotion or gel for styling or treating the hair, in the form of a lotion or gel for brushing or blow-waving, in the form of a hair lacquer, permanent waving composition, colorant or bleach for the hair. The preparation having light-protection properties may comprise adjuvants, such as interface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

Formulations comprising the BiOCl pigment according to the invention may furthermore comprise at least one constituent selected from the group absorbents, astringents, antimicrobial substances, antioxidants, antiperspirants, antifoaming agents, antidandruff active compounds, antistatics, binders, biological additives, bleaches, chelating agents, deodorants, emollients, emulsifiers, emulsion stabilisers, dyes, humectants, film formers, fillers, fragrances, flavours, insect repellents, preservatives, anticorrosion agents, cosmetic oils, solvents, oxidants, plant constituents, buffer substances, reducing agents, surfactants, propellant gases, opacifiers, UV filters, UV absorbers, denaturing agents, aloe vera, avocado oil, coenzyme Q10, green tea extract, viscosity regulators, perfumes, vitamins, enzymes, trace elements, proteins, carbohydrates, organic pigments, inorganic pigments, including effect pigments.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The present invention is explained in greater detail by the following examples, but is not intended to be restricted thereto.

### EXAMPLES

### Example 1

In a Turbula mixer (WAB), 100 g of dried flake-form BiOCl pigments having a particle size <20 μm and a thickness of 60-80 nm, D₅₀=15 μm and D₈₀=20 μm (Malvern Mastersizer MS 3000) are mixed with 2 g of sucrose (powdered sugar) for 30 min at a frequency of 34 rpm. The carbon coating is carried out in a fluidised-bed reactor (DI=100 mm) at a reaction temperature of 250° C. The fluidisation gas used is N₂. The fluidisation velocity corresponds to 10 times the value of the expected theoretical minimum fluidisation velocity of 2 mm/s. The carbon coating is carried out under inert conditions for 30 min. The fluidised BiOCl particle bed is then cooled to room temperature, likewise under inert gas. The carbon coating is subsequently sieved.

The amorphous-carbon-coated BiOCl pigment obtained is distinguished by a metallic silver-grey lustre.

### Example 2

In a Turbula mixer (WAB), 100 g of dried flake-form BiOCl pigments having a particle size <20 μm and a thickness of 50-80 nm, D₅₀=15 μm and D₈₀=20 μm (Malvern Mastersizer MS 3000) are mixed with 1 g of sucrose (powdered sugar) for 30 min at a frequency of 72 rpm. The carbon coating is carried out in a fluidised-bed reactor (DI=100 mm) at a reaction temperature of 200° C. The fluidisation gas used is N₂. The fluidisation velocity corresponds to 10 times the value of the expected theoretical minimum fluidisation velocity of 2 mm/s. The carbon coating is carried out under inert conditions for 30 min. The fluidised BiOCl particle bed is then cooled to room temperature, likewise under inert gas. The carbon coating is subsequently sieved.

The pigment from Example 2 exhibits a darker metallic silver-grey lustre compared with Example 1.

### Example 3

In a Turbula mixer (WAB), 100 g of flake-form BiOCl pigments of the RonaFlair® LF-2000 type (Merck, BiOCl pigment with D₅₀=8-20 μm, D₈₀<35 μm, thickness 200-500 nm) are mixed with 3 g of D-fructose for 30 min at a frequency of 200 rpm. The carbon coating is carried out in a fluidised-bed reactor (DI=100 mm) at a reaction temperature of 220° C. The fluidisation gas used is N₂. The fluidisation velocity corresponds to 10 times the value of the expected theoretical minimum fluidisation velocity of 2 mm/s. The carbon coating is carried out under inert conditions for 20 min. The fluidised BiOCl particle bed is then cooled to room temperature, likewise under inert gas. The carbon coating is subsequently sieved.

A dark pigment with a silver-white lustre is obtained.

### Example 4

In a Turbula mixer (WAB), 100 g of flake-form BiOCl pigments of the RonaFlair® B-50 type (2-35 μm, D₅₀=9-15 μm, D₈₀ 2-35 μm, thickness 200-500 nm) are mixed with 2.5 g of glucose for 30 min at a frequency of 150 rpm. The carbon coating is carried out in a fluidised-bed reactor (DI=100 mm) at a reaction temperature of 150° C. The fluidisation gas used is N₂. The fluidisation velocity corresponds to 10 times the value of the expected theoretical minimum fluidisation velocity of 2 mm/s. The carbon coating is carried out under inert conditions for 20 min. The fluidised BiOCl particle bed is then cooled to room temperature, likewise under inert gas. The carbon coating is subsequently sieved.

A dark pigment with a silver-white lustre is obtained.

### Example 5

In a Turbula mixer (WAB), 100 g of flake-form BiOCl pigments of the RonaFlair® ESQ type (product from Merck KGaA having a particle size of 2-35 μm, D₅₀=11-17 μm, D₈₀ 2-35 μm, thickness 200-500 nm) are mixed with 5 g of lactose for 40 min at a frequency of 45 rpm. The carbon coating is carried out in a fluidised-bed reactor (DI=100 mm) at a reaction temperature of 280° C. The fluidisation gas used is N₂. The fluidisation velocity corresponds to 10 times the value of the expected theoretical minimum fluidisation velocity of 2 mm/s. The carbon coating is carried out under inert conditions for 20 min. The fluidised BiOCl particle bed is then cooled to room temperature, likewise under inert gas. The carbon coating is subsequently sieved.

A very dark pigment with a silver lustre is obtained.

### Example 6

In a Turbula mixer (WAB), 100 g of flake-form BiOCl pigments of the RonaFlair® Fines type (product from Merck KGaA having a particle size of 2-35 μm, D₅₀=9-15 μm, D₈₀ 2-35 μm, thickness 50-100 nm) are mixed with 1 g of glucose for 30 min at a frequency of 60 rpm. The carbon coating is carried out in a fluidised-bed reactor (DI=100 mm) at a reaction temperature of 250° C. The fluidisation gas used is N₂. The fluidisation velocity corresponds to 10 times the value of the expected theoretical minimum fluidisation velocity of 2 mm/s. The carbon coating is carried out under inert conditions for 30 min. The fluidised BiOCl particle bed is then cooled to room temperature, likewise under inert gas. The carbon coating is subsequently sieved.

A pale pigment with a silver lustre is obtained.

### Example 7

100 g of flake-form BiOCl pigments of the RonaFlair® MTU type (product from Merck KGaA having a particle size of 2-35 μm, thickness 500 700 nm) are mixed with 2.5 g of sucrose (powdered sugar) in a fluidised-bed reactor (DI=100 mm) for 30 min at the minimum fluidisation velocity. The carbon coating is carried out in the fluidised-bed reactor at a reaction temperature of 180° C. The fluidisation gas used is N₂. The fluidisation velocity corresponds to 10 times the value of the expected theoretical minimum fluidisation velocity of 2 mm/s. The carbon coating is carried out under inert conditions for 30 min. The fluidised BiOCl particle bed is then cooled to room temperature, likewise under inert gas. The carbon coating is subsequently sieved.

A pale pigment with a silver lustre is obtained.

The pinhole-free, amorphous carbon coating of the BiOCl flakes is demonstrated using confocal RAMAN spectroscopy and with the aid of transmission electron microscopy (TEM).

The BiOCl pigments coated with amorphous carbon are distinguished by increased stability, by a metallic silver lustre and high hiding power and can be dispersed and re-dispersed very well in application media.

### USE EXAMPLES

### Use Example 1: Paint

20.35 g of BiOCl pigment from Example 1 are incorporated into 679.65 g of a commercially available water-based base coat (Mipa WBC000, Mipa).

The drying is carried out at 80° C. for 5 min. The pigmented paint is applied to a metal panel and subsequently overcoated with 805 g of 2-component clear coat (Mipa CC4, Mipa).

The drying is carried out at room temperature for 15 min and subsequently at 80° C. for 5 min.

### Use Example 2: Paint

20.35 g of BiOCl pigment from Example 2 are incorporated into 679.65 g of a commercially available water-based base coat (Mipa WBC000, Mipa).

The drying is carried out at 80° C. for 5 min. The pigmented paint is applied to a metal panel and subsequently overcoated with 805 g of 2-component clear coat (Mipa CC4, Mipa).

The drying is carried out at room temperature for 15 min and subsequently at 80° C. for 5 min.

The liquid-metal effect of the painted panels is investigated in accordance with Equation 1 using a BYK-mac i from BYK Gardner GmbH, a multi-angle colour measuring instrument.

The liquid-metal effect of a coating system is measured and evaluated by means of the liquid index LI, which is defined as the ratio of the flop index FI and the graininess G and is calculated as follows:

$$LI = \frac{FI}{G} = \frac{\frac{{( {L}_{15 °}^{*} - {L}_{110 °}^{*} )}^{1 , 11}}{{L}_{45}^{* 0 , 86}}}{G} . \qquad ( I )$$

FI describes the lightness difference L* at flat (L*(15°)) and steep (L*(110°)) viewing angles. The coating texture is described by the quantity G (graininess). Layer-like application of paint, irrespective of the use in an automotive paint or a cosmetic coating, only exhibits the liquid-metal effect in the case where FI is greater than 21. At the same time, G must have values less than three, so that the LI in the case of a liquid-metal finish is always greater than seven (see measurement values for Example 1 in the table).

| | FI | G | LI |
|---|---|---|---|
| | (flop index) | (graininess) | (liquid index) |
| Coated BiOCl | 21.7 | 2.4 | 8.9 |
| pigment from | | | |
| Example 1 | | | |
| Uncoated BiOCl | 18.5 | 2.8 | 6.6 |
| pigment from | | | |
| Example 1 | | | |

The pigments according to the invention from Examples 1 and exhibit a pronounced liquid-metal effect in the paint.

### Use Example 3—Eye Shadow

| | | |
|---|---|---|
| Raw | INCI | |
| material | (CTFA) | wt.-% |

Phase A

| | | | |
|---|---|---|---|
| Pigment from | (1) | | 30.00 |
| Example 1 | | | |
| Parteck ® | (1) | TALC | 10.00 |
| LUB Talc | | | |

Phase B

| | | | |
|---|---|---|---|
| RonaCare ® AP | (1) | BIS-ETHYLHEXYL | 0.50 |
| | | HYDROXYDIMETHOXY | |
| | | BENZYLMALONATE | |
| Oxynex ® K | (1) | PEG-8, TOCOPHEROL, | 0.10 |
| liquid | | ASCORBYL PALMITATE, | |
| | | ASCORBIC ACID, | |
| | | CITRIC ACID | |
| all-rac-alpha- | (1) | TOCOPHERYL ACETATE | 0.50 |
| Tocopheryl acetate | | | |
| Parteck ® | (1) | STEARIC ACID | 3.00 |
| LUB STA 50 | | | |
| SP Crodamol PMP | (2) | PPG-2 MYRISTYL | 30.90 |
| MBAL-LQ-(MH) | | ETHER PROPIONATE | |
| Syncrowax HGLC | (2) | C18-36 ACID | 10.00 |
| | | TRIGLYCERIDE | |
| Miglyol ® 812 N | (3) | CAPRYLIC/CAPRIC | 8.00 |
| | | TRIGLYCERIDE | |
| Syncrowax HRC | (2) | TRIBEHENIN | 3.00 |
| Ganex ™ V-216 | (4) | PVP/HEXADECENE | 2.00 |
| | | COPOLYMER | |
| Sunflower oil, | (5) | HELIANTHUS ANNUUS SEED | 1.00 |
| refined | | OIL (HELIANTHUS ANNUUS | |
| | | (SUNFLOWER) SEED OIL) | |
| Sensiva ® PA | (6) | PHENETHYL ALCOHOL, | 1.00 |
| 20 | | ETHYLHEXYL GLYCERIN | |

Preparation:

Heat phase B to 80° C. until all constituents have melted. Cool to 65° C. and add the constituents of phase A with stirring. Transfer the material into the desired container at 65° C. Cool to room temperature.

Manufacturers:

(2) Croda

(3) 101 Oleo GmbH (4) Ashland

(5) Gustav Heess GmbH (6) Schulke & Mayr GmbH

### Use Example 4—Eye Shadow Gel

Phase A

| Raw | Source of | | |
|---|---|---|---|
| material | supply | INCI | wt.-% |
| Pigment from | Merck KGaA/ | | 15.00 |
| Example 2 | Rona ® | | |
| Micronasphere ® M | Merck KGaA/ | MICA, SILICA | 8.00 |
| | Rona ® | | |
| Carbopol | Noveon | ACRYLATES/C10-30 | 0.40 |
| Ultrez 21 | | ALKYL ACRYLATE | |
| | | CROSSPOLYMER | |
| Citric acid | Merck KGaA/ | CITRIC ACID | 0.00 |
| monohydrate | Rona ® | | |
| Water | | AQUA (WATER) | to 100 |

Phase B

| Raw | Source of | | |
|---|---|---|---|
| material | supply | INCI | wt.-% |
| Glycerin | Merck KGaA/ | GLYCERIN | 3.00 |
| | Rona ® | | |
| Preservative | | | q.s. |
| Triethanolamine | | TRIETHANOLAMINE | 0.70 |
| Water | | AQUA (WATER) | 13.00 |

Phase C

| Raw | Source of | | |
|---|---|---|---|
| material | supply | INCI | wt.-% |
| Lubrajel DV | | PROPYLENE GLYCOL, | 5.00 |
| | | POLYGLYCERYL | |
| | | METHACRYLATE | |

Preparation:

Disperse the pigment and the Micronasphere® in the water of phase A. Acidify using a few drops of citric acid in order to reduce the viscosity, scatter in the Carbopol with stirring. When dissolution is complete, slowly stir in the pre-dissolved phase B and subsequently phase C. Finally, adjust the pH to between 7.0-7.5.

### Use Example 5—Lipstick

Phase A

| Raw | Source of | | |
|---|---|---|---|
| material | supply | INCI | wt.-% |
| Pigment from | | | 12.00 |
| Example 2 | | | |
| Ronastar ® | Merck KGaA/ | Calcium Aluminum | 3.00 |
| Purple Sparks | Rona ® | Borosilicate, CI77891 | |
| | | (Titanium Dioxide), | |
| | | Silica, Tin Oxide | |

Phase B

| Raw | Source of | | |
|---|---|---|---|
| material | supply | INCI | wt-% |
| Beeswax | Merck KGaA/ | Cera Alba | 8.75 |
| | Rona ® | (Beeswax) | |
| Paracera | Paramelt | COPERNICIA | 5.25 |
| C44 | | CERIFERA | |
| | | (CARNAUBA WAX), | |
| | | CERESIN | |
| Adeps Lanae | Henry Lamotte | LANOLIN | 3.50 |
| | GmbH | | |
| Isopropyl | Cognis GmbH | Isopropyl | 5.60 |
| myristate | | Myristate | |
| Viscous | Merck KGaA/ | PARAFFINUM | 2.10 |
| paraffin | Rona ® | LIQUIDUM | |
| | | (MINERAL OIL) | |
| Castor oil | Henry Lamotte | RICINUS | 59.65 |
| | GmbH | COMMUNIS | |
| | | (CASTOR OIL) | |
| Oxynex ® | Merck KGaA/ | PEG-8, | 0.05 |
| K liquid | Rona ® | TOCOPHEROL, | |
| | | ASCORBYL | |
| | | PALMITATE | |
| | | ASCORBIC ACID, | |
| | | CITRIC ACID | |
| Propyl | Merck KGaA/ | PROPYLPARABEN | 0.10 |
| 4-hydroxy- | Rona ® | | |
| benzoate | | | |

Preparation:

The constituents of phase Bare heated to 75° C. and melted. The pigments of phase Aare added, and everything is stirred well. The lipstick composition is then stirred for 15 minutes in the casting apparatus held at a temperature of 65° C. The homogeneous melt is poured into the casting mould prewarmed to 5500. The moulds are subsequently cooled and the cold castings are removed. After warming to room temperature, the lipsticks are briefly flame-treated.

### Use Example 6—Nail Varnish

| Raw | Source of | | |
|---|---|---|---|
| material | supply | INCI | wt.-% |
| Pigment from | | | 2.00 |
| Example 3 | | | |
| Nailsyn ® | Merck KGaA/ | CI 77163 (Bismuth | 1.00 |
| Sterling 60 | Rona ® | Oxychloride), Butyl | |
| Silver | | Acetate, | |
| | | Nitrocellulose, | |
| | | Isopropyl Alcohol, | |
| | | Ethyl Acetate, | |
| | | Stearnalkonium | |
| | | Hectorite | |
| Thixotropic | Durlin/ | BUTYL ACETATE, | 97.00 |
| nail varnish | Bergerac NC | ETHYL ACETATE | |
| base 155 | | NITROCELLULOSE, | |
| | | ACETYL TRIBUTYL | |
| | | CITRATE, PHTHALIC | |
| | | ANHYDRIDE/ | |
| | | TRIMELLITIC | |
| | | ANHYDRIDE/GLYCOLS | |
| | | COPOLYMER, | |
| | | ISOPROPYL ALCOHOL, | |
| | | STEARALKONIUM | |
| | | HECTORITE, ADIPIC | |
| | | ACID/FUMARIC | |
| | | ACID/PHTHALIC | |
| | | ACID/TRICYCLODECANE | |
| | | DIMETHANOL | |
| | | COPOLYMER, | |
| | | CITRIC ACID | |

Preparation:

The pigment and the Nailsyn® Sterling 60 Silver are weighed out together with the varnish base, mixed well by hand using a spatula and subsequently stirred at 1000 rpm for 10 min.

### Use Example 7—Soap

| Raw | Source of | | |
|---|---|---|---|
| material | supply | INCI | wt-% |
| Pigment from | | | 1.50 |
| Example 1 | | | |
| Ronastar ® | Merck KGaA/ | Calcium Aluminum | 0.50 |
| Noble Sparks | Rona ® | Borosilicate, Silica, | |
| | | CI 77891 (Titanium | |
| | | Dioxide), Tin Oxide | |
| Transparent | Jean Charles | SODIUM PALMATE, | 98.00 |
| soap base | (USA) | SODIUM LAURETH | |
| | | SULFATE, SODIUM | |
| | | STEARATE, | |
| | | SODIUM MYRISTATE, | |
| | | SODIUM COCOYL | |
| | | ISETHIONATE, | |
| | | TRIETHANOLAMINE, | |
| | | AQUA (WATER), | |
| | | GLYCERIN, SORBITOL, | |
| | | PROPYLENE GLYCOL, | |
| | | FRAGRANCE | |

Preparation:

All constituents are mixed homogeneously.

The entire disclosure[s] of all applications, patents and publications, cited herein and of corresponding German Patent Application No. 102019008593.0, filed Dec. 11, 2019, is [are] incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A coated BiOCl pigment, which comprises a flake-form BiOCl pigment coated on the surface with an amorphous carbon layer.

2. The BiOCl pigment according to claim 1, wherein the flake-form BiOCl pigment has a particle size of 1-30 μm.

3. The BiOCl pigment according to claim 1, wherein the BiOCl pigment has a thickness of 50-100 nm.

4. The BiOCl pigment according to claim 1, wherein the BiOCl pigment has a volume-weighted D₅₀ value of <25 μm (determined using a Malvern).

5. The BiOCl pigment according claim 1, wherein the BiOCl pigment has an aspect ratio (ratio of length or width to thickness) of 10-600.

6. The BiOCl pigment according to claim 1, wherein the amorphous carbon layer has a geometrical layer thickness of 1 to 5 nm.

7. The BiOCl pigment according to claim 1, wherein the proportion of the amorphous carbon layer, based on the weight of the coated BiOCl pigment, is 0.5-5% by weight.

8. A process for the preparation of the coated BiOCl pigment according to claim 1, which comprises covering the flake-form BiOCl pigment as substrate with a layer of amorphous carbon in a reactor in a stream of carrier gas at a temperature of 150-400° C. in the presence of one or more carbon-containing compounds by pyrolytic decomposition of the carbon-containing compound(s).

9. The process according to claim 8, wherein one or more carbon-containing compound is acetone, ethine or a pulverulent compound selected from the group consisting of mono-, di- or trisaccharides.

10. The process according to claim 8, wherein one or more carbon-containing compound is fructose, glucose, dextrose, galactose, xylose, mannose, lactose, sucrose, maltose or a mixture of the said compounds.

11. The process according to claim 8, wherein the flake-form BiOCl pigment is kept in motion in the reactor.

12. A paint, coating, industrial coating, automotive paint, printing ink, paper, plastic, film, cosmetic, button paste or RADAR-permeable coating formulation or a dry preparation for pigment preparations, comprising a coated BiOCl pigment according to claim 1.

13. A three-coat paint system, comprising a coated BiOCl pigment according to claim 1.

14. A composition comprising the coated BiOCl pigment according to claim 1 and a further component.

15. The composition according to claim 14, wherein the further component is a component used in an industrial coating or automotive paint.

16. The composition according to claim 14 wherein the further component is a component used in a cosmetic.

17. The composition according to claim 14, wherein the further component is at least one constituent selected from the group consisting of absorbents, astringents, antimicrobial substances, antioxidants, antiperspirants, antifoaming agents, antidandruff active compounds, antistatics, binders, biological additives, bleaches, chelating agents, deodorants, emollients, emulsifiers, emulsion stabilisers, dyes, humectants, film formers, fillers, fragrances, flavours, insect repellents, preservatives, anticorrosion agents, cosmetic oils, solvents, oxidants, plant constituents, buffer substances, reducing agents, surfactants, propellant gases, opacifiers, UV filters, UV absorbers, denaturing agents, aloe vera, avocado oil, coenzyme Q10, green tea extract, viscosity regulators, perfumes, vitamins, enzymes, trace elements, proteins, carbohydrates, organic pigments and inorganic pigments.
